(19) United States
(12) Patent Application Publication
(10) Pub. No.: US 2021/0196863 A1
(43) Pub. Date: Jul. 1, 2021

(54) HYDROGEL FOR ENDOGENOUS NEUROPROGENITOR CELL RECRUITMENT

(71) Applicant: THE REGENTS OF THE UNIVERSITY OF CALIFORNIA, Oakland, CA (US)

(72) Inventors: Tatiana Segura, Durham, NC (US); Lina R. Nih, Los Angeles, CA (US); Elias Sideris, Pasadena, CA (US)

(73) Assignee: THE REGENTS OF THE UNIVERSITY OF CALIFORNIA, Oakland, CA (US)

(21) Appl. No.: 16/075,100

(22) PCT Filed: Feb. 1, 2017
(86) PCT No.: PCT/US2017/016028

§ 371 Date: Aug. 2, 2018

Related U.S. Application Data
(60) Provisional application No. 62/290,372, filed on Feb. 2, 2016.

(51) Int. Cl. A61L 27/52; A61L 27/20; A61L 27/54; A61L 27/58

(52) U.S. Cl. CPC A61L 27/52; A61L 27/20; A61L 27/54; A61L 2400/06; A61L 2300/414; A61L 2300/42; A61L 2300/622; A61L 27/58

(57) ABSTRACT

A hydrogel material for the treatment of stroke or other brain injury includes a collection of hyaluronic acid-based microgel particles comprising one or more network crosslinker components, wherein the hyaluronic acid-based microgel particles, when exposed to an endogenous annealing agent (e.g., Factor XIIIa), links the hyaluronic acid-based microgel particles together in situ to form a covalently-stabilized scaffold of microgel particles having interstitial spaces therein. The hydrogel material may be injected into a stroke cavity and was shown to promote brain tissue repair by promoting the recruitment of neural stem cells to the injured site and reducing the post-stroke inflammatory response.

### RELATED APPLICATION

This Application claims priority to U.S. Provisional Patent Application No. 62/290,372 filed on Feb. 2, 2016, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with Government support under R01NS079691, awarded by the National Institutes of Health. The Government has certain rights in the invention.

### TECHNICAL FIELD

The technical field generally relates to therapeutic hydrogels and in particular hydrogels that are injected into brain tissue to promote cellular infiltration and neurogenesis.

### BACKGROUND

Stroke is currently the most prevalent and devastating neurological disease. Up to 800,000 people experience a first-time stroke (more if recurrent strokes are factored in) and few completely recover. Deficits in the control of limb function contribute most to the inability of stroke patients to regain function. Because mortality from stroke is declining but incidence is not, stroke is transforming into a chronic, disabling disease. To date, no therapeutics exists after the first four and one-half hours after the stroke onset, aside from rest and physical therapy. Following stroke, a large influx of astrocytes and microglia releasing pro-inflammatory cytokines leads to massive inflammation and glial scar formation, affecting brain tissue's ability to repair itself. Brain repair in stroke subjects generally occurs through the recruitment of endogenous neuronal progenitor cells to the damaged site and brain plasticity. Neural progenitor cells (NPCs) are cells that have the capacity to differentiate into all neural cell types that are found in the mammalian brain. These include neurons and other neural cells that form the interconnected network that defines brain tissue. Although the promotion of their migration towards injury sites is an endogenous process activated after trauma or disease, these NPC cells rarely reach the boundary of the injured site. First, NPCs are often found far from the lesion (infarct) or the peri-infarct tissue (i.e., around the lesion) and may not reach the stroke site if it is distant to the subventricular zone niche where NPCs migrate from. Second, NPCs are highly sensitive to their environment and the majority of them die after leaving their niche, which reduced dramatically the total number of cells in migration. Finally, the post-stroke brain creates a thick scar around the wound to protect the surrounding healthy tissue from the massive inflammation and cell death that follows stroke. This scar forms a physical barrier around the stroke site and prevents NPCs from infiltrating it and creating new neuronal tissue within the stroke cavity.

### SUMMARY

In one embodiment, a microporous hydrogel is injected into the brain tissue to promote the recruitment of endogenous cells into the stroke cavity. The microporous hydrogel, in one embodiment, is formed as an interconnected scaffold of microgel particles that are annealed or otherwise linked to one another. Interstitial pores, spaces, and voids are formed within the scaffold that supports cell adhesion and infiltration. In one aspect of the invention, the microgel particles are formed from hyaluronic acid-based microgel particles. In another aspect of the invention, the microgel particles that form the scaffold that is delivered to the brain are polydispersed with respect to size (e.g., diameter). In another embodiment, the microgel particles that form the scaffold are formed from hyaluronic acid-based microgel particles and are polydispersed with respect to size. A polydisperse, hyaluronic acid-based microporous hydrogel formed from a network of particles has been shown to significantly reduce the inflammatory response following stroke while increasing pen-infarct vascularization. The microporous hydrogel also results in an increased NPC migration into the stroke site.

In another embodiment, a hyaluronic acid-based microporous hydrogel is injected into brain tissue of a mammal (e.g., human or animal) to promote the recruitment of endogenous cells into the stroke cavity that created as a result of the stroke. The hydrogel includes a collection of hyaluronic acid-based microgel particles comprising one or more network crosslinker components, wherein the hyaluronic acid-based microgel particles, when exposed to an endogenous or exogenous annealing agent, links the hyaluronic acid-based microgel particles together in situ to form a covalently-stabilized scaffold of microgel particles having interstitial spaces therein that promote the adhesion and recruitment of NPCs. The microgel particles are injected into the compartmentalized cavity that naturally forms following stroke, the pen-infarct area, or the brain surface. The microgel particles may be optionally loaded with cells such as NPCs, trophic factors, and/or growth factors to promote tissue repair and healing.

In one embodiment, a hydrogel material for the treatment of stroke or other brain injury includes a collection of hyaluronic acid-based microgel particles of non-uniform size comprising one or more crosslinker components for linking different microgel particles, wherein the hyaluronic acid-based microgel particles, when exposed to an endogenous or exogenous annealing agent, links the hyaluronic acid-based microgel particles together in situ to form a covalently-stabilized scaffold of microgel particles having interstitial spaces therein.

In another embodiment, a method of treating stroke in a subject includes injecting a collection of hyaluronic acid-based microgel particles of non-uniform size comprising one or more crosslinker components for linking different microgel particles into a stroke cavity, wherein the hyaluronic acid-based microgel particles, when exposed to an endogenous or exogenous annealing agent, links the hyaluronic acid-based microgel particles together in situ to form a scaffold of microgel particles having interstitial spaces therein.

### BRIEF DESCRIPTION OF THE DRAWINGS

FIG. 1A illustrates a hydrogel material formed from microgel particles that has been injected into brain tissue post stroke and annealed to form a microporous scaffold.

FIG. 1B illustrates a sectional view of a mouse brain section having a stroke cavity therein that has been injected with microgel particles according to one embodiment of the invention.

FIG. 1C illustrates one exemplary method of synthesizing a hyaluronic acid-acrylate (HA-Ac) polymer.

FIG. 1D illustrates one exemplary method of modifying the HA-Ac polymer with a cell adhesion peptide and K and Q peptides used crosslink different microgel particles using a dicysteine-containing matrix metalloproteinase degradable peptide.

FIG. 2A schematically illustrates a microfluidic device used to generate the microgel particles from a solution of HA-Ac and the matrix metalloproteinase degradable peptide.

FIG. 2B schematically illustrates another embodiment of a microfluidic device that has an additional pair of outer channels downstream of the pinching oil channels.

FIG. 3 illustrates three different cross-sectional views of a healthy brain, stroke brain, and stroke brain injected with a MAP gel (containing microgel particles).

FIG. 4A illustrates a graph of hyaluronic acid-based bead or microgel particle size (diameter; μm) as a function of frequency percentage that were produced using the microfluidic device described herein.

FIG. 4B illustrates a graph of the total void fraction of a scaffold formed using the hyaluronic acid-based microgel particles described herein.

FIG. 4C illustrates a graph of the pore size (A=area μm²; d=diameter μm) of a scaffold formed using the hyaluronic acid-based microgel particles described herein.

FIG. 5 illustrates a graph of the Young's modulus of a scaffold formed using the hyaluronic acid-based microgel particles described herein in compression calculated using Instron mechanical tests.

FIG. 6 illustrates a graph showing scar thickness (um) obtained using GFAP staining for the No Gel, npore (nanopore), and MAP gels (containing microgel particles).

FIG. 7 illustrates a graph showing GFAP (astrocytes) peri-infarct area (%) obtained using GFAP staining for the No Gel, npore (nanopore), and MAP gels (containing microgel particles).

FIG. 8 illustrates a graph showing GFAP (astrocytes) infarct area (%) obtained using GFAP staining for the No Gel, npore (nanopore), and MAP gels (containing microgel particles).

FIG. 9 illustrates a graph showing GFAP (astrocytes) infiltration (um) obtained using GFAP staining for the No Gel, npore (nanopore), and MAP gels (containing microgel particles).

FIG. 10A illustrates fluorescent stained GFAP (astrocytes) images of the stroke area of the brain for the No Gel condition.

FIG. 10B illustrates fluorescent stained GFAP (astrocytes) images of the stroke area of the brain for the MAP Gel condition (i.e., MAP gel injected into stroke cavity).

FIG. 10C schematically illustrates the same anatomical space of FIG. 10A (No Gel condition).

FIG. 10D schematically illustrates the same anatomical space of FIG. 10B (MAP Gel condition).

FIG. 11A illustrates fluorescent stained Iba-1 (microphages/microglia) images of the stroke area of the brain for the No Gel condition.

FIG. 11B illustrates fluorescent stained Iba-1 (microphages/microglia) images of the stroke area of the brain for the MAP Gel condition (i.e., MAP gel injected into stroke cavity).

FIG. 11C schematically illustrates the same anatomical space of FIG. 11A (No Gel condition).

FIG. 11D schematically illustrates the same anatomical space of FIG. 11B (MAP Gel condition).

FIG. 12 illustrates a graph showing Iba-1 (microphages/microglia) infarct area (%) obtained using Iba-1 staining for the No Gel, npore (nanopore), and MAP gels (containing microgel particles).

FIG. 13 illustrates a graph showing Iba-1 (microphages/microglia) pen-infarct area (%) obtained using Iba-1 staining for the No Gel, npore (nanopore), and MAP gels (containing microgel particles).

FIG. 14 illustrates a graph obtained using Glut1 (blood vessel) fluorescent images showing increased vasculature in the MAP gel in the pen-infarct area.

FIG. 15 illustrates a graph obtained using NF200 (axons) fluorescent images showing increased neuronal axons in the in the MAP gel in the pen-infarct area as compared to the No Gel state but no difference when compared to the npore (nanopore) condition.

FIG. 16 illustrates a graph of cell number at the ipsilateral ventricle wall for the No Gel, npore (nanopore), and MAP gels (containing microgel particles). *, *** and **** indicate P<0.05, P<0.001 and P<0.0001, respectively (Anova 1 way, Tukey's post-hoc test).

FIG. 17 illustrates a graph of cell number at the migrating path for the No Gel, npore (nanopore), and MAP gels (containing microgel particles).

FIG. 18 illustrates a graph of migrating distance (μm) at the migrating path for the No Gel, npore (nanopore), and MAP gels (containing microgel particles).

FIG. 19 illustrates a graph of the positive area for DCX (NPC) signal in the stroke site for the No Gel, npore (nanopore), and MAP gels (containing microgel particles) conditions. **** indicates P<0.0001 (Anova 1 way, Tukey's post-hoc test).

### DETAILED DESCRIPTION OF THE ILLUSTRATED EMBODIMENTS

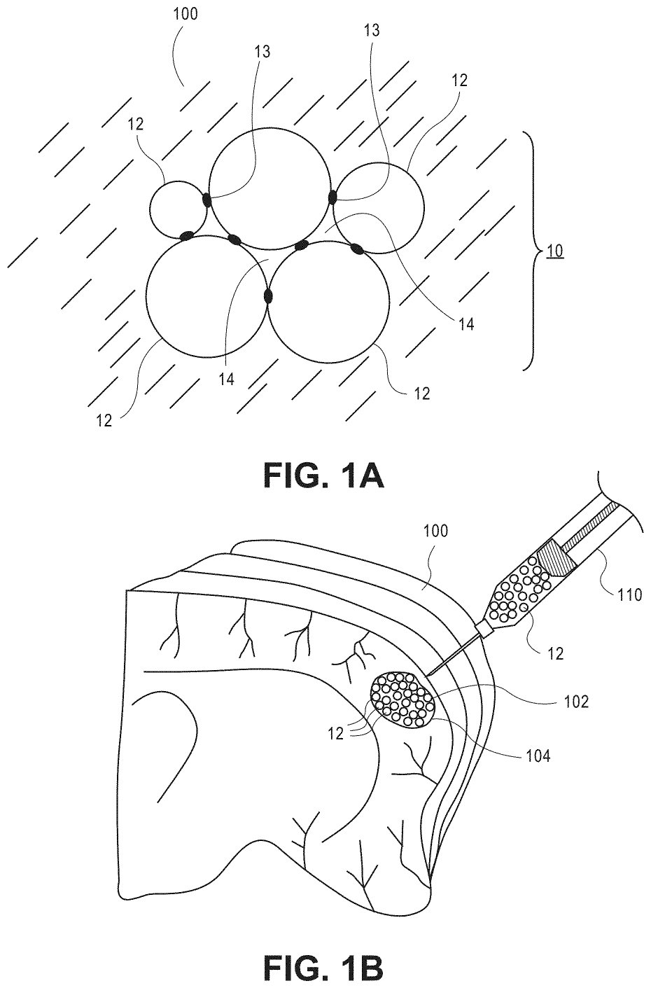

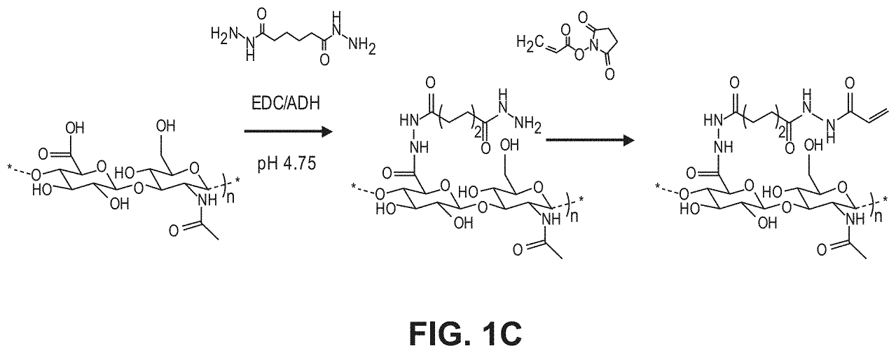

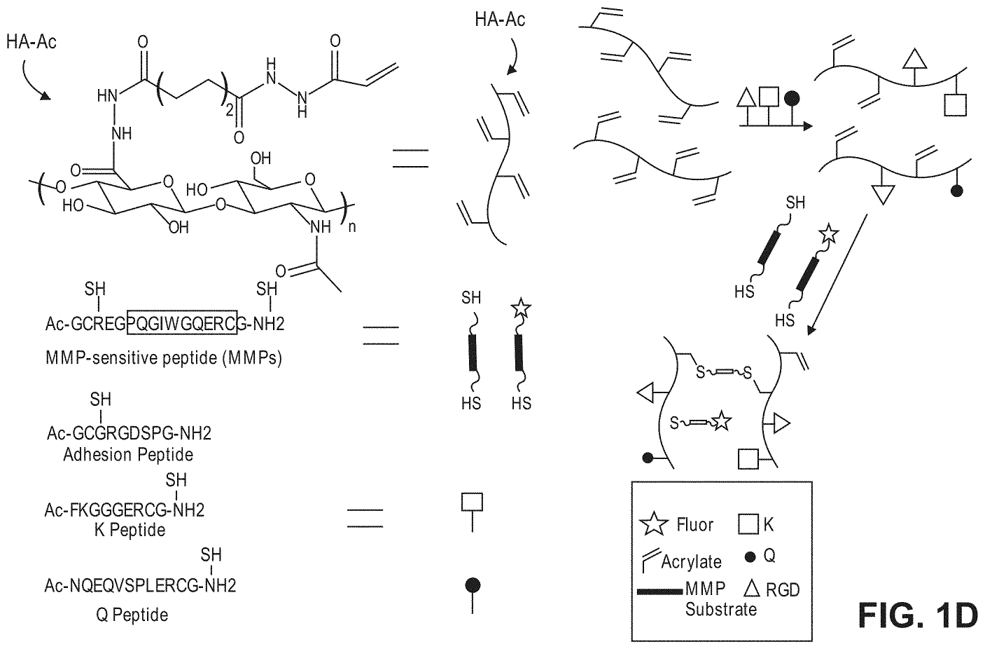

FIG. 1A illustrates a portion of the formed three dimensional scaffold 10 that is formed by a plurality of annealed microgel particles 12 that are injected or otherwise delivered into brain tissue 100 of a mammal (e.g., human or animal). The microgel particles 12 are secured to one another via annealing connections 13 as illustrated in FIG. 1A. FIG. 1A illustrates the microgel particles 12 having a spherical shape. However, it should be understood that the microgel particles 12 may have non-spherical shapes as well. The scaffold 10 includes interstitial spaces therein 14 that are voids that form micropores within the larger scaffold 10. The network of interstitial spaces or voids 14 located between annealed microgel particles 12 have dimensions and geometrical profiles that permit the infiltration, binding, and growth of NPC cells. As explained herein, the microgel particles 12 may be delivered as a slurry or mixture using a delivery device such as a syringe or other applicator commonly known to deliver fluids to a delivery site within tissue and specifically within brain tissue 100.

The delivery site described herein is a stroke cavity 102 such as that illustrated in FIG. 1B that naturally forms after stroke. After initial cell death that follows a stroke, the clearance of debris in the lesion leaves a compartmentalized cavity 102 that can accept a large volume of the microgel particles 12 without further damaging the surrounding healthy parenchyma. This stroke cavity 102 is situated directly adjacent to the peri-infarct tissue area 104, the region of the brain that undergoes the most substantial repair and recovery, meaning that any therapeutic delivered to the cavity 102 will have direct access to the tissue target for repair. In addition to being deliverable to the stroke cavity 102, the microgel particles 12 may also be transplanted in the pen-infarct area 104, or the brain surface 100. In one optional embodiment, the microgel particles 12 may be mixed with cells (e.g., NPCs), trophic factors, and/or growth factors such as BDNF (Brain Derived-Neurotrophic Factor), BMP-4 (Bone Morphogenic Protein-4), ciliary neurotrophic factor, platelet derived growth factor, epidermal growth factor, or VEGF (Vascular Endothelial Growth Factor) prior to injection in order to promote tissue repair and healing through the activation of endogenous neurogenesis or angiogenesis.

In one aspect of the subject matter described herein, the microporous gel system uses microgel particles 12 have diameter dimensions within the range from about 20 μm to about 120 μm with the microgel particles 12 that form the scaffold 10 being non-uniform in size. The term “non-uniform” when used in this context is meant to indicate that the there is a variation in the size of the individual microgel particles 12 that form the scaffold 10. Some of the microgel particles 12 may be “small” (yet still within the diameter size range of about 20 μm to about 120 μm) while other microgel particles 12 may be large “large” (yet still within the diameter size range of about 20 μm to about 120 μm). The above description describes a binary system of microgel particles 12 but it should be understood that the scaffold 10 may be formed from a variety of sizes of microgel particles 12—not simply a binary grouping of sizes. The non-uniform nature of the size of the microgel particles 12 is believed to result from the higher viscosity of the hyaluronic acid as compared to other polymers such as poly(ethylene glycol) that have been used. While not being bound to a particular theory or hypothesis, it is believed that the non-uniform nature of the scaffold 10 contributes to the recruitment of NPCs into the lesion site.

As explained herein, in a particular preferred embodiment, the microgel particles 12 are made from hyaluronic acid (HA) in which hyaluronic acid was modified through carbodiimide chemistry to introduce crosslinkable acrylamide groups (HA-Ac) on the HA backbone. FIG. 1C illustrates one exemplary method of synthesizing a hyaluronic acid-acrylate (HA-Ac) polymer. In this method, hyaluronic acid was modified with adipic dihydrazide (ADH) after activating the carboxylic acid with carbodiimide. The HA-ADH polymer was dialyzed, lyophilized and then further modified with NHS-Acrylate to create the hyaluronic acid-acrylate (HA-Ac) polymer. The HA-Ac was purified and lyophilized to create the final product.

Specifically, HA (60,000 Da, Genzyme Corporation, Cambridge, Mass.) (2.0 g, 5.28 mmol) was dissolved in water mixed with adipic dihydrazide (ADH, 18.0 g, 105.5 mmol) with 1-ethyl-3-(dimethylaminopropyl) carbodiimide hydrochloride (EDC, 4.0 g, 20 mmol) with pH adjusted to 4.75. This mixture was allowed to react overnight to form hydrazide-modified hyaluronic acid (HA-ADH). The next day purification was performed via dialysis (8000 MWCO) in deionized water for 2 days. The HA-ADH was then lyophilized. HA-ADH (1.9 g) was dissolved in 4-(2-hydroxyethyl)-1-piperazine ethanesulfonic acid (HEPES) buffer (10 mM HEPES, 150 mM NaCl, 10 mM EDTA, pH 7.4) and mixed with N-acryloxysuccinimide (NHS-AM, 1.33 g, 4.4 mmol) and allowed to react overnight. The next day purification was performed via dialysis against deionized water for 2 days, and HA-Acrylate (HA-Ac) was lyophilized. The product was analyzed with ¹H NMR (D20) and the percent modification (14%) was determined by dividing the multiplet peak at δ=6.2 (cis and trans acrylate hydrogens) by the singlet peak at δ=1.6 (singlet peak of acetyl methyl protons in HA monomer). The HA-Ac was stored under Argon at −20° C. until used.

With reference to FIG. 1D, the HA-Ac polymer was modified with three peptides (adhesion peptide RGD (Ac-RGDSPGERCG-NH₂ [SEQ ID NO: 1]) and two Factor XIIIa substrates: Ac-FKGGERCG-NH₂ [SEQ ID NO: 2] (K-peptide), and Ac-NQEQVSPLGGERCG-NH₂ [SEQ ID NO: 3] (Q-peptide)), and then crosslinked through Michael-type addition using a dicysteine-containing matrix metalloproteinase degradable peptide Ac-GCREGPQGIWGQERCG-NH2 [SEQ ID NO: 4]. The highlighted region of SEQ ID NO: 4 in FIG. 1D refers to the target sequence of the matrix metalloproteinase enzyme. The crosslinking takes place in an oil-coated aqueous droplet generated in a microfluidic device 50 as illustrated in FIG. 1E, resulting in the formation of non-uniform sized beads or microgels (μgels), that will serve microgel particles 12 that are function as the building blocks to form the three dimensional scaffold 10 within the brain tissue 100. Particle-based gel systems have been described previously as Microporous Annealed Particle or “MAP” hydrogels, although these have not been utilized in brain tissue to recruit NPCs in response to a stroke.

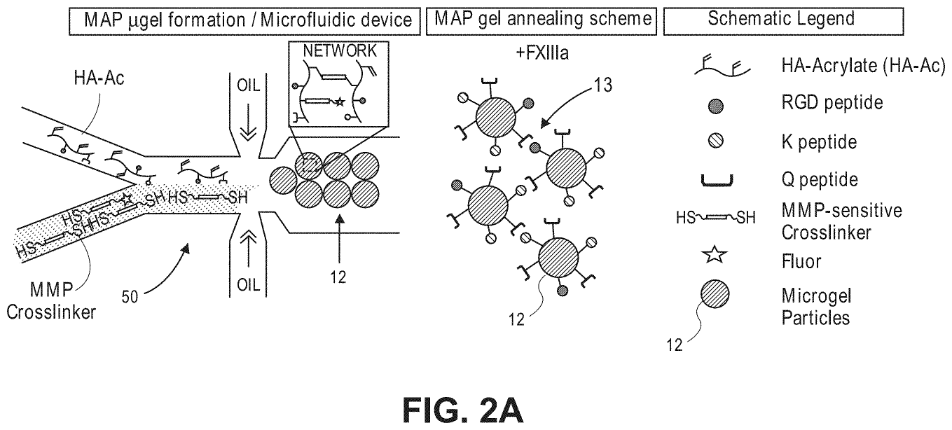

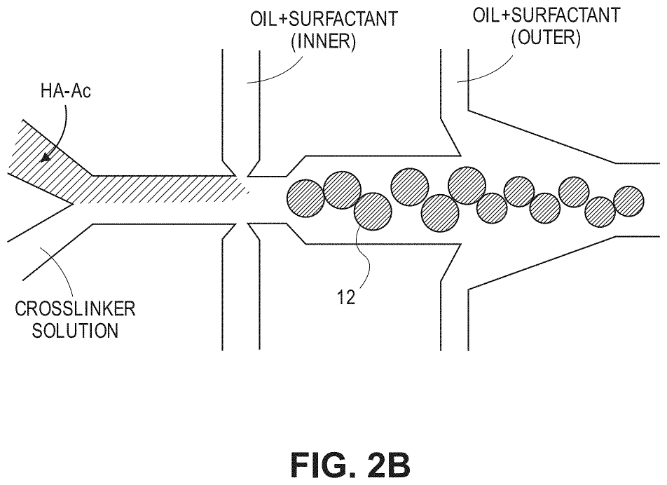

FIG. 2A schematically illustrates the microfluidic device 50 that is used to generate the microgel particles 12 in spherical droplet shapes. The microfluidic device 50 is a four inlet, one outlet microfluidic droplet generator previously reported in Griffin et al., Accelerated wound healing by injectable gel scaffolds assembled form annealed building blocks, Nature Materials, 14, 737-744 (2015), which is incorporated by reference herein. Two inlets were reserved for the “pinch” oil (1% v/v span-80 in heavy mineral oil) and “outer” oil (5% v/v span-80 in heavy mineral oil) while the other two inlets allowed the HA-Ac solution and the crosslinker solution to be mixed immediately before the “pinch” point. Note that FIG. 2A does not illustrate the “outer” oil channels intersecting with the main channel; this aspect of additional outer oil channels is seen in FIG. 2B which schematically illustrates the microfluidic device 50 used to generate the microgel particles 12 using inner oil channels for pinching particles 12 or droplets and outer oil channels. The HA-Ac solution was freshly prepared before each run by first dissolving HA-Ac in 0.3 M triethanolamine (TEOA) pH 8.8 at 7% w/v. This solution was then used to dissolve three thiol-containing pendent peptides: K-peptide [SEQ ID NO: 2], Q-peptide NH₂ [SEQ ID NO: 3], and RGD [SEQ ID NO: 1] at 500 μM, 500 μM, and 1000 μM, respectively. The thiol-containing pendent peptides had been previously combined and lyophilized to a powder containing 0.2 μ-moles of K-peptide, 0.2 μ-moles of Q-peptide, and 0.4 μ-moles of RGD so that 400 μL of the HA-Ac solution could be prepared and loaded into the 1 mL Hamilton Gas-tight syringe after a 30-minute incubation at 37° C. to pre-reaction the thiol-containing pendent peptides with the HA-Ac. Meanwhile, the crosslinker solution was prepared by dissolving the di-thiol matrix metalloproteinase (MMP) sensitive linker peptide [SEQ ID NO: 4] in distilled water at 7.8 mM.

For experiments described herein that utilized fluorescent reporting, the di-thiol matrix metalloproteinase (MMP) sensitive linker was reacted with 10 μM Alexa-Fluor 488-maleimide (Life-Technologies) for five minutes. Of course, for therapeutic or clinical applications there is no need for fluorescent reporting so this aspect may be omitted. The crosslinker solution was then loaded into another 1 mL Hamilton Gas-tight syringe, total volume of 400 μL. Two syringe pumps were used to separately control the flow rates of the oils and the gel precursor solutions. The gel precursor solutions were co-flowed at a 1:1 volume to make the final microgel droplets (or microspheres) and left overnight at 25° C. to crosslink (this reaction is known as Michael-type addition) to form the crosslinked microgel particles 12. Table 1 below illustrates the flow rates and device parameters used to make the microgel particles 12.

TABLE 1

| | | |
|---|---|---|
| Aqueous Flow | 0.8-1.0 | μL/min |
| Pinch Oil Flow | 6.0-8.0 | μL/min |
| Outer Oil Flow | 6.0-8.0 | μL/min |
| Aqueous Channel Width | 25 | μm |
| Pinch Channel Width | 10 | μm |

The final microgel composition was 3.5 wt % HA-AM, 250 μM K-peptide, 250 μM Q-peptide, 500 μM RGD, 5 μM Alexa-Fluor 488-maleimide (for fluorescent reporting experiments), and 3.9 mM crosslinker (thiol:AM is 0.8). The microgel particles 12 are then transferred to micro-centrifuge tubes and HEPES buffer saline (pH 7.4 containing 10 μM CaCl₂) was added to each tube. The tubes were then centrifuged at 18,000 G's for five minutes, allowing for a separation between the pelleted microgel particles 12 and the oil plus surfactant. This supernatant is aspirated and the procedure above was repeated until all the oil and surfactant was removed from the microgel particles 12 (˜5 to 6 times).

To anneal the microgel particles 12 to one another to form the three dimensional scaffold 10 in the brain tissue 100, a hydrated solution containing the microgel particles 12 is pelleted by centrifuging at 18,000 G and discarding the supernatant. In this particular embodiment, FXIII and Thrombin was used as the exogenous annealing agent to anneal the microgel particles 12 to each other. Specifically, 5 U/mL of FXIII and 1 U/mL of Thrombin were combined with the pelleted microgel particles 12 before injection into the brain (an endogenous agent such as FXIIIa or activated FXIII could also be used). The mixture is loaded into a delivery device 110 such as syringe as seen in FIG. 2B that has a needle that can be used to precisely deliver the desired volume of microgel particles 12 to the stroke cavity 102, the pen-infarct area 104, or the brain surface 100. The microgel particles 12 will then anneal to one another over the next 60-90 minutes to form the scaffold 10 at the site of application.

During clinical use, the patient or subject will typically be first given a scan such as a magnetic resonance imaging (MRI) scan to localize the location and volume of the stroke site 102. The first three days (e.g., at about five days) after stroke are associated with a massive inflammatory response where cellular debris resulting from cell death in the damaged site are cleared by specialized inflammatory cells (microphages/microglia) leaving behind an empty cavity. The specific localization of both the infarct (stroke cavity) and the peri-infarct areas are determined with 3 dimensional intra-cerebral coordinates (x, y and z). To access the stroke cavity 102, a hole or access passageway is drilled in the subject's skull (e.g., craniotomy) adjacent to the site of the stroke. Most strokes occur in the cerebral cortex or outer layer of brain tissue which can be then be readily accessed after the formation of the craniotomy. The delivery device 110 is then inserted into the craniotomy and the microgel particles 12 are then delivered to the stroke cavity 102. In one embodiment, the delivery device 110 may be mounted on an armature or moveable support structure so that the delivery device 110 may be positioned properly to deliver the microgel particles to the stroke cavity 102. This may include an automated system that is mounted for x, y, and z directions movement using actuators, servos, or the like so that placement and injection is accomplished automatically. Of course, in an alternative embodiment, the delivery device 110 may be manipulated manually to deliver the microgel particles 12.

The mechanical properties of the microporous hydrogel scaffold 10 can be modulated by changing the mechanical properties of the building blocks, which are controlled though the percent polymer and the crosslinking ratio. Importantly, a microporous hydrogel scaffold 10 with a stiffness of around 300-350 Pa (shear modulus), which is similar to brain cortex, can be generated. Further, the microgel particle 12 slurry mixture is injectable and can take the shape of a void, recess, or defect (e.g., stroke cavity 102). The amount of hyaluronic acid may vary but may be around 3.5% (on a weight percentage basis). The annealed solid scaffold with voids may be degradable such that it degrades over time but survives long enough so that NCPs can enter and travel within the microporous interstitial spaces 14 and promote neurogenesis and the healing process.

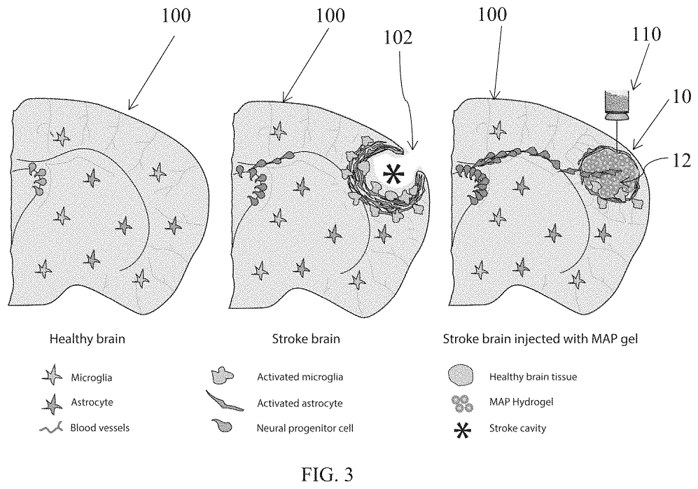

FIG. 3 illustrates a schematic representation of healthy brain, stroke brain, and stroke brain that has been injected with the HA-based microporous hydrogel scaffold 10 described herein. Astrocytes, microglia, and vasculature are illustrated in the healthy brain. The stroke brain illustrates the stroke cavity 102 as well as activated astrocytes and microglia as well as NPCs. The stroke brain that has been injected with the microporous hydrogel scaffold 10 illustrates a syringe 110 injecting the microgel particle 12 slurry mixture into the stroke cavity 102 to form the microporous hydrogel scaffold 10. With reference to FIGS. 1B and FIG. 3, HA based microgel particles 12 are injected several days post stroke onset and gelled in situ to form a bulk scaffold 10 within the stroke cavity 102. Ischemic stroke occurs when an obstruction blocks blood flow in a blood vessel. After delivery of the microgel particles 12 to the site of injection, individual microgel particles 12 are annealed together by Factor XIIIa, an enzyme found naturally in the blood (or Factor XIII and Thrombin are added to the slurry of microgel particles 12 just prior to injection which creates activated Factor XIII or Factor XIIIa). A bond is formed between the K and Q peptides in the presence of Factor XIIIa, resulting in a fully annealed scaffold 10. The interconnected microporosity occurs from the imperfect stacking of the microgel particles 12. Moreover, the elastic modulus of the scaffold is around 900-1000 Pa, matching the stiffness of the cortex. The HA-based microporous hydrogel scaffold 10 reduces brain inflammation post stroke, by promoting astrocyte infiltration into the stroke cavity rather than scar formation and reducing the total number of reactive microglia within the infarct. These events lead to an environment that allows neuroprogenitor cell migration into the material and stroke cavity.

While the microgel particles 12 described herein in one preferred embodiment utilize hyaluronic acid (HA) modified through carbodiimide chemistry to introduce crosslinkable acrylamide groups (HA-Ac) on the HA backbone; other hydrogel materials may also be used in some embodiments. For example, the microgel particles 12 may be made from a hydrophilic polymer, amphiphilic polymer, synthetic or natural polymer (e.g., poly(ethylene glycol) (PEG), poly(propylene glycol), poly(hydroxyethylmethacrylate), hyaluronic acid (HA), gelatin, fibrin, chitosan, heparin, heparan, and synthetic versions of HA, gelatin, fibrin, chitosan, heparin, or heparin).

In one embodiment, the microgel particles 12 are made from any natural (e.g., modified HA) or synthetic polymer (e.g., PEG) capable of forming a hydrogel. In one or more embodiments, a polymeric network and/or any other support network capable of forming a solid hydrogel construct may be used. Preferably, such materials are biodegradable over a period of elapsed time. Examples of suitable biocompatible and biodegradable supports include: natural polymeric carbohydrates and their synthetically modified, crosslinked, or substituted derivatives, such as gelatin, agar, agarose, crosslinked alginic acid, chitin, substituted and crosslinked guar gums, cellulose esters, especially with nitrous acids and carboxylic acids, mixed cellulose esters, and cellulose ethers; natural polymers containing nitrogen, such as proteins and derivatives, including crosslinked or modified gelatins, and keratins; vinyl polymers such as poly(ethyleneglycol)acrylate/methacrylate/vinyl sulfone/maleimide/norbornene/allyl, polyacrylamides, polymethacrylates, copolymers and terpolymers of the above polycondensates, such as polyesters, polyamides, and other polymers, such as polyurethanes; and mixtures or copolymers of the above classes, such as graft copolymers obtained by initializing polymerization of synthetic polymers on a preexisting natural polymer. A variety of biocompatible and biodegradable polymers are available for use in therapeutic applications; examples include: polycaprolactone, polyglycolide, polylactide, poly(lactic-co-glycolic acid) (PLGA), and poly-3-hydroxybutyrate. Methods for making networks from such materials are well-known.

In one or more embodiments, the microgel particles 12 further include covalently attached chemicals or molecules that act as signaling modifications that are formed during microgel particle 12 formation. Signaling modifications includes the addition of, for example, adhesive peptides, extracellular matrix (ECM) proteins, and the like. Functional groups and/or linkers can also be added to the microgel particles 12 following their formation through either covalent methods or non-covalent interactions (e.g., electrostatic charge-charge interactions or diffusion limited sequestration). Crosslinkers are selected depending on the desired degradation characteristic. For example, crosslinkers for the microgel particles 12 may be degraded hydrolytically, enzymatically, or the like. In one particular preferred embodiment, the crosslinker is a matrix metalloprotease (MMP)-degradable crosslinker such as that described herein.

Examples of these crosslinkers are synthetically manufactured or naturally isolated peptides with sequences corresponding to MMP-1 target substrate, MMP-2 target substrate, MMP-9 target substrate, random sequences, Omi target sequences, Heat-Shock Protein target sequences, and any of these listed sequences with all or some amino acids being D chirality or L chirality. In another embodiment, the crosslinker sequences are hydrolytically degradable natural and synthetic polymers consisting of the same backbones listed above (e.g., heparin, alginate, poly(ethyleneglycol), polyacrylamides, polymethacrylates, copolymers and terpolymers of the listed polycondensates, such as polyesters, polyamides, and other polymers, such as polyurethanes).

In another embodiment, the crosslinkers are synthetically manufactured or naturally isolated DNA oligos with sequences corresponding to: restriction enzyme recognition sequences, CpG motifs, Zinc finger motifs, CRISPR or Cas-9 sequences, Talon recognition sequences, and transcription factor-binding domains. Any of the crosslinkers from the listed embodiments one are activated on each end by a reactive group, defined as a chemical group allowing the crosslinker to participate in the crosslinking reaction to form a polymer network or gel, where these functionalities can include: cysteine amino acids, synthetic and naturally occurring thiol-containing molecules, carbene-containing groups, activated esters, acrylates, norborenes, primary amines, hydrazides, phosphenes, azides, epoxy-containing groups, SANPAH containing groups, and diazirine containing groups.

In one embodiment, the chemistry used to generate microgel particles 12 allows for subsequent annealing and scaffold formation through radically-initiated polymerization. This includes chemical-initiators such as ammonium persulfate combined with Tetramethylethylenediamine. Alternatively, photoinitators such as Irgacure® 2959 or Eosin Y together with a free radical transfer agent such as a free thiol group (used at a concentration within the range of 10 μM to 1 mM) may be used in combination with a light source that is used to initiate the reaction as described herein. One example of a free thiol group may include, for example, the amino acid cysteine, as described herein. Of course, peptides including a free cysteine or small molecules including a free thiol may also be used. Another example of a free radical transfer agent includes N-Vinylpyrrolidone (NVP).

Alternatively, Michael and pseudo-Michael addition reactions, including α,β-unsaturated carbonyl groups (e.g., acrylates, vinyl sulfones, maleimides, and the like) to a nucleophilic group (e.g., thiol, amine, aminoxy) may be used to anneal microgel particles 12 to form the scaffold. In another alternative embodiment, microgel particle 12 formation chemistry allows for network formation through initiated sol-gel transitions including fibrinogen to fibrin (via addition of the catalytic enzyme thrombin).

Functionalities that allow for particle-particle annealing are included either during or after the formation of the microgel particles 12. In one or more embodiments, these functionalities include α,β-unsaturated carbonyl groups that can be activated for annealing through either radical initiated reaction with α,β-unsaturated carbonyl groups on adjacent particles or Michael and pseudo-Michael addition reactions with nucleophilic functionalities that are either presented exogenously as a multifunctional linker between particles or as functional groups present on adjacent particles. This method can use multiple microgel particle 12 population types that when mixed form a scaffold 10. For example, microgel particle of type X presenting, for example, nucleophilic surface groups can be used with microgel particle type Y presenting, for example, α,β-unsaturated carbonyl groups. In another embodiment, functionalities that participate in Click chemistry can be included allowing for attachment either directly to adjacent microgel particles 12 that present complimentary Click functionalities or via an exogenously presented multifunctional molecule that participates or initiates (e.g., copper) Click reactions.

The annealing functionality can include any previously discussed functionality used for microgel crosslinking that is either orthogonal or similar (if potential reactive groups remain) in terms of its initiation conditions (e.g., temperature, light, pH) compared to the initial crosslinking reaction. For example if the initial crosslinking reaction consists of a Michael-addition reaction that is temperature dependent, the subsequent annealing functionality can be initiated through temperature or photoinitiation (e.g., Eosin Y, Irgacure®). As another example, the initial microgel particles 12 may be photopolymerized at one wavelength of light (e.g., ultraviolent with Irgacure®), and annealing of the microgel particles 12 occurs at the same or another wavelength of light (e.g., visible with Eosin Y) or vice versa. Besides annealing with covalent coupling reactions, annealing moieties can include non-covalent hydrophobic, guest/host interactions (e.g., cyclodextrin), hybridization between complementary nucleic acid sequences or nucleic acid mimics (e.g., protein nucleic acid) on adjoining microgel particles 12 or ionic interactions. An example of an ionic interaction would consist of alginate functionality on the microgel particle surfaces that are annealed with Ca2+. So-called “A+B” reactions can be used to anneal microgel particles 12 as well. In this embodiment, two separate microgel particle 12 types (type A and type B) are mixed in various ratios (between 0.01:1 and 1:100 A:B) and the surface functionalities of type A react with type B (and vice versa) to initiate annealing. These reaction types may fall under any of the mechanisms listed herein.

Experimental

HA-based microporous hydrogel was synthesized using the three stages described herein. First, the hyaluronic acid was modified through carbodiimide chemistry to introduce crosslinkable acrylamide groups (HA-Ac) on the HA backbone. Second, this polymer was modified with three peptides (adhesion peptide RGD (Ac-RGDSPGERCG-NH₂ [SEQ ID NO: 1]) and two Factor XIIIa substrates: Ac-FKGGERCG-NH₂ [SEQ ID NO: 2] (K-peptide), and Ac-NQEQVSPLGGERCG-NH₂ [SEQ ID NO: 3] (Q-peptide)), and then crosslinked through Michael-type addition using a dicysteine-containing matrix metalloproteinase degradable peptide Ac-GCREGPQGIWGQERCG-NH2 [SEQ ID NO: 4]. The crosslinking takes place in an oil-coated aqueous droplet generated in the microfluidic device illustrated in FIGS. 2A and 2B.

These microgel particles 12 were purified to remove oil and surfactants using repeated washing with buffer and centrifugation. Third, the microgel particles 12 were linked to each other with factor XIIIa to form an annealed solid with void spaces. In these experiments, the HA-based hydrogel was labeled during microgel particle 12 generation using a maleimide-containing fluorophore such that the MAP scaffold can be imaged with standard confocal microscopy after sectioning.

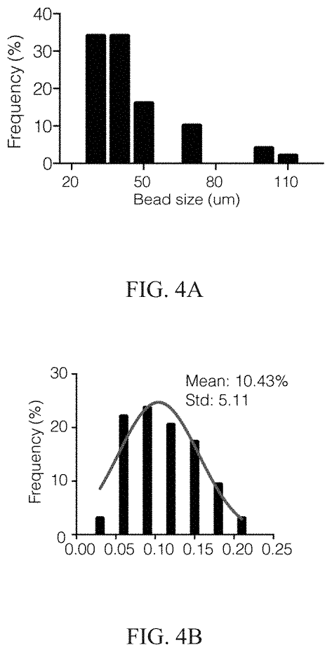

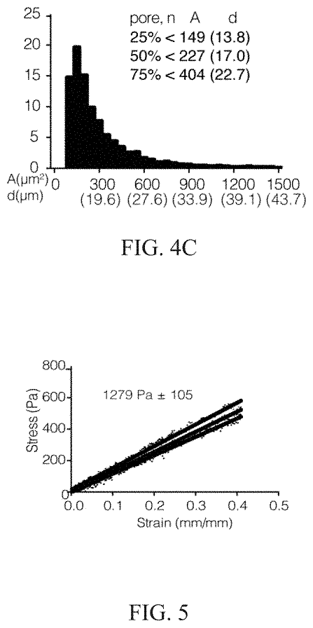

Using the microfluidic device 50 of FIGS. 2A and 2B, HA-Ac solution pre-reacted with the K, Q, and RGD peptides was flowed through one channel and MMP sensitive crosslinker was flowed in the second channel. These two channels merge to form the hydrogel precursor solution, which is quickly pinched by heavy mineral oil containing 1% surfactant to form droplets. The flow regime used (1 μL/min for the aqueous flow and 8 μL/min for the oil flow) produced a range of microgel particle 12 sizes with an average microgel particle 12 diameter of 45 μm as seen in FIG. 4A. FIG. 4B illustrates a graph of the total void fraction of the microgel scaffold 10. The mean void fraction of the MAP scaffold is 10.43% meaning that 89.67% of the scaffold volume is hydrogel. FIG. 4C illustrates a graph of the pore sizes of the MAP scaffold. The median pore diameter is 17 μm.

To determine the mechanical properties of annealed scaffolds purified microgel particles 12 were pelleted by centrifuging at 18,000 G and discarding the supernatant to form a concentrated solution of microgel particles 12. Five (5) U/mL of FXIII and one (1) U/mL of Thrombin were combined in the presence of 10 mM Ca²⁺ with the pelleted microgel particles 12 before injection and allowed to incubate at 37° C. for 90 minutes between two slides (1 mm thickness) surface coated with Sigmacote (Sigma-Aldrich). The mechanical testing on the hydrogel scaffolds was done using a 5500 series Instron. After annealing, the scaffolds were allowed to swell in HEPES buffer saline for 4 hours at room temperature. A 2.5N load cell with a 3.12 mm tip in diameter was used at a compression strain rate of 1 mm/min and the hydrogel scaffold was indented 0.8 mm or 80% of its total thickness. Instron mechanical testing on the resulting annealed scaffold revealed a Young's Modulus of 1279 Pa as seen in FIG. 5, which closely matches the stiffness of native cortex tissue of the brain.

It was first studied whether the hydrogel injection and immune reaction toward HA-based hydrogel scaffolds to ensure that they will not further aggravate the brain damage after stroke. Brain ischemic strokes in the sensorimotor cortex were created using a middle cerebral artery occlusion (MCAo) model where a brain artery is cauterized and sectioned to stop blood flow in the designated area. A total of 6 μL HA MAP (i.e., HA-Ac hydrogel scaffolds made from microgel particles 12) were injected into the cavity five days post stroke and animals were sacrificed 10 days post injection, and compared with a negative control where mice were injected with the same volume of an HA nanoporous (HA NP) bulk hydrogel containing pores at the nano scale (but not annealed microgel particles 12).

Animal procedures were performed in accordance with the U.S. National Institutes of Health Animal Protection Guidelines and the University of California Los Angeles Chancellor's Animal Research Committee. A permanent cortical stroke was induced by a middle cerebral artery occlusion (MCAo) on young adult C57BL/6 male mice (8-12 weeks) obtained from Jackson laboratories. Under anesthesia, a small craniotomy was made over the left parietal cortex where an anterior branch of the distal middle cerebral artery was then exposed, electrocoagulated, cut to be permanently occluded, and bilateral jugular veins were clamped for 15 min. Five days following stroke surgery, microgel particles 12 with FXIIIa were loaded into a Hamilton syringe (Hamilton Reno, NV) connected to a pump and 6 μL of microgel particles 12 were injected into the stroke cavity using a 30-gauge needle at stereotaxic coordinates 0.26 mm anterior/posterior (AP), 3 mm medial/lateral (ML), and 1 mm dorsal/ventral (DV) with an infusion speed of 1 μL/min. The needle was withdrawn from the mouse brain five minutes after the injection to allow for annealing of the microgel particles 12. Ten days following the hydrogel transplantation, mice were sacrificed via transcardial perfusion of 0.1 M PBS followed by 40 mL of 4 (w/v) % PFA. The brains were isolated and post-fixed in 4% PFA overnight and submerged in 30 (w/v) % sucrose solution for 24 hours.

Tangential cortical sections of 30 μm-thickness were sliced using a cryostat and directly mounted on gelatin-subbed glass slides for immunohistological staining of GFAP (glial fibrillary acidic protein, Abcam, Cambridge, Mass., USA) for astrocytes, Iba1 (ionized calcium binding adaptor molecule, Abcam, Cambridge, Mass., USA) for microglial cells, Glut-1 (Glucose Transporter-1, Abcam, Cambridge, Mas., USA) for endothelial cells, NF200 (Neurofilament 200, Abcam, Cambridge, Mass., USA) for axonal processes, DCX (doublecortin, Abcam, Cambridge, Mass., USA) for NPCs, Ki67 (Abcam, Cambridge, Mass., USA) for proliferating cells, and DAPI (1:500 Invitrogen) for nuclei. Primary antibodies (1:100) were incubated overnight at 4° C. and secondary antibodies (1:1000) were incubated at room temperature for two hours.

A Nikon C2 confocal microscope was used to take fluorescent images. Analyses were performed on microscope images of three (3) coronal brain levels at +0.80 mm, −0.80 mm and −1.20 mm according to bregma, which consistently contained the cortical infarct area. Each image represents a maximum intensity projection of 10 to 12 Z-stacks, 1 um apart, captured at a 20× magnification with a Nikon C2 confocal microscope using the NIS Element software.

A group of mice with stroke but no gel injection (No Gel) was used as a negative control. HA-Ac hydrogel injection into the stroke cavity 102 did not cause brain swelling or deformation and filled the entire cavity, indicating that the gel injection and hydrogel annealing in situ did not affect the brain structure. Next, the inflammatory response to hydrogels was analyzed by assessing astrogliosis and microgliosis 10-days post injection. Astrogliosis was assessed through GFAP (Glial Fibrillary Acidic Protein) staining by measuring the astrocytic scar thickness and total percent positive signal in the infarct (within the stroke) and peri-infarct (around the stroke) regions. The thickness of scar was measured on the ischemic boundary zone within the ipsilateral hemisphere on three sections stained for GFAP. The proliferating NPC cell count and migrating distance were measured on the ipsilateral hemisphere and represents the total number of double labeled Dcx/Ki67 positive cells present on the ventricle wall and migrating toward the infarcted zone, the maximum migration distance of NPCs was measured between the upper corner of the ipsilateral wall on the corpus callosum and the furthest Dcx/Ki67 positive cell on the migrating path toward the stroke site.

The endothelial (Glut-1), astrocytic (GFAP) and inflammation (microglia) (Iba-1) positive area in the infarct and peri-infarct areas were quantified in 4 to 8 randomly chosen regions of interest (ROI of 0.3 mm²). In each ROI, the positive area was measured using pixel threshold on 8-bit converted images using ImageJ (Image J v1.43, Bethesda, Maryland, USA) and expressed as the area fraction of positive signal per ROI (%). Values were then averaged across all ROI and sections, and expressed as the average positive area per animal.

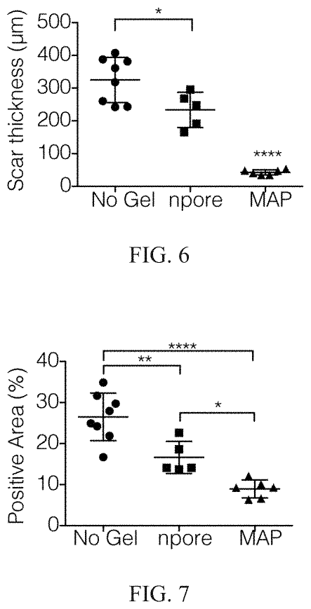

A drastic decrease in the astrocytic scar thickness surrounding the MAP gel was observed when compared to the nano-porous (npore) gel and the No gel condition (No Gel) as seen in FIG. 6. The scar in the MAP condition was only 43±8 μm thick while in the nano-porous gel and No gel conditions, the scar was 234±54 and 325±69 μm thick, respectively, almost a 6× difference.

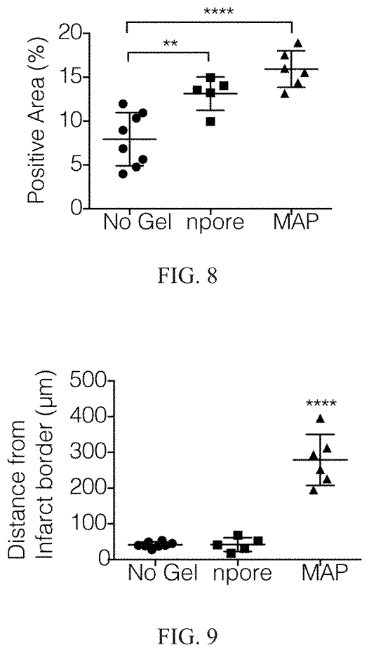

This led to a lower percentage of astrocytes in the peri-infarct area of the MAP gel condition as seen in FIG. 7. These results show that introducing a hydrogel decreases the scar thickness, while introducing microporosity in the hydrogel drastically reduces the scar thickness. However, analysis of the GFAP signal within the stroke cavity revealed a statistical increase for both the MAP condition and the nano-porous condition compared to the No gel control as seen in FIG. 8. This observation was surprising as substantial infiltration by GFAP positive cells of the stroke cavity has not been previously observed. Further analysis showed that MAP gel injection promoted astrocyte infiltration into the infarct with an average infiltration length of 279±71 μm compared to only 42±19 μm in the nano-porous condition causing a higher percentage of astrocytes to occupy the infarct area in the MAP gel condition as seen in FIG. 9. Interestingly these differences in astrocyte infiltration are due to the topography of the scaffold alone as the MAP and nano-porous scaffolds have the exact same biochemical signals and bulk moduli.

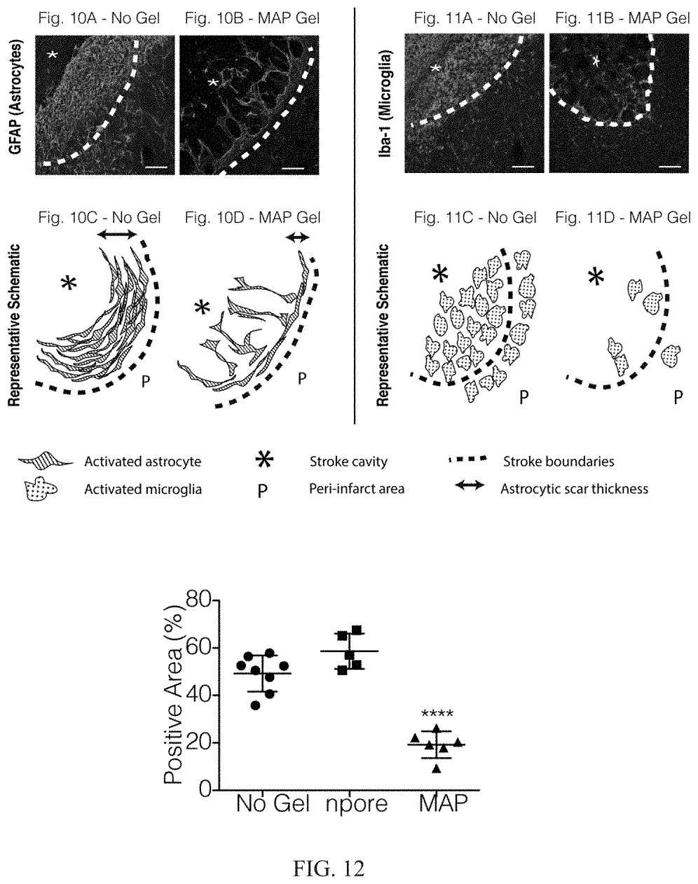

FIGS. 10A and 10B illustrate fluorescent stained GFAP images of the stroke area of the brain for both the No Gel and MAP Gel conditions. Stained GFAP images (astrocytes) are used identify the formation of the scar after stroke. FIGS. 10C and 10D illustrated below FIGS. 10A and 10B, respectively, schematically illustrate the same anatomical space (with reference to stroke cavity and pen-infarct area) and further illustrate activated astrocytes, the corresponding astrocytic scar thicknesses. One can clearly see a much reduced scar thickness in the MAP Gel (FIGS. 10B and 10D) treated stroke as compared to the No Gel state. Further, activated astrocytes were surprisingly found to have infiltrated the stroke cavity. FIGS. 11A and 11B illustrate fluorescent Iba-1 images of the same stroke area of the brain for both the No Gel and MAP Gel conditions. Stained (Ionized calcium binding adaptor molecule-1) Iba-1 images (microglia) are used identify the infiltration of inflammatory cells in the stroke area. FIGS. 11C and 11D illustrated below FIGS. 11A and 11B, respectively, schematically illustrate the same anatomical space (with reference to stroke cavity and peri-infarct area) and further illustrate activated microglia. In the No Gel condition, activated microglia are found to have infiltrated the stroke cavity (FIG. 11C) while in the stroke brain that received the MAP gel, very few activated microglia are seen (FIG. 11D).

In order to protect the healthy tissue from the nearby lesion area (stroke), star-shaped glial cells, astrocytes, elongate cytosolic processes surround the damaged site, forming the astrocytic scar. The long-term persisting pen-lesion scar is known to act as a physical barrier to tissue regeneration by blocking the way to axonal, vascular and neuronal infiltration. After the initial cell death in stroke, the activation and recruitment of microphage-like cells called microglia allows for the clearance of debris in the lesion, leaving a compartmentalized cavity that can accept a large volume transplant without damaging further the surrounding healthy parenchyma. Both phenomena are known to create a toxic environment that prevents pro-repair cells from growing in the stroke area and repairing the lost tissue. As seen in FIGS. 10B, 10D, 11B, 11D, the injection of the MAP gel within the stroke area induces a dramatic remodeling of the astrocytic scar, by reducing its thickness and allowing astrocytes to infiltrate the damaged area where they play a role of guidance for pro-repair cells. The inflammatory presence of microglia is also reduced with a drastic reduction of the number of cells present in and around the stroke, decreasing the toxicity of the peri-lesion environment, a first step essential to create a cell-friendly environment.

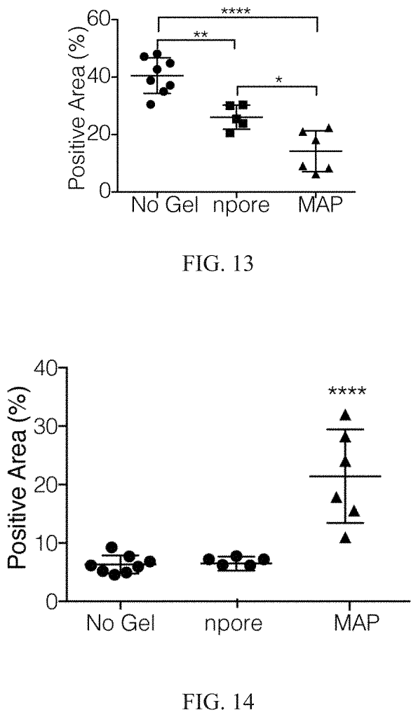

As noted above, significant differences in microglial response, assessed by the Iba-1 signal were observed. The percent area occupied by the microglia was significantly reduced in both the infarct and pen-infarct areas in the MAP gel condition compared to the nano-porous and No gel conditions (FIGS. 12 and 13). While only 19% of the infarct area was positive for microglia in the MAP condition, 58% of the infarct area in the nano-porous condition and 50% in the No gel conditions were positive for Iba-1. Taken together this shows that both astrogliosis and microgliosis are significantly reduced in animals injected with MAP gels resulting in reduced scar thickness and decreased reactive microglia.

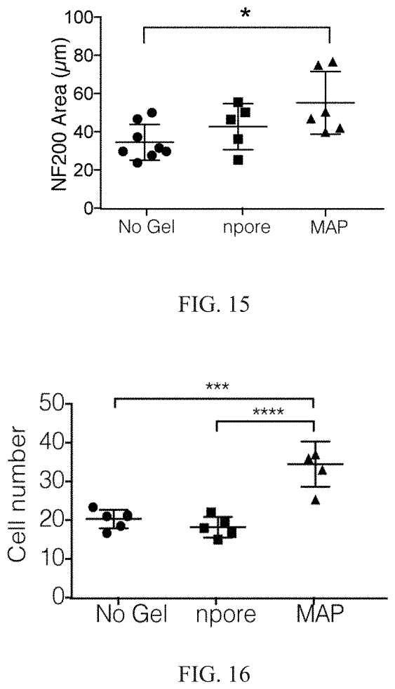

After the inflammatory response was examined, cells within the infarct of the MAP condition were observed that were not stained for astrocytes or microglia. Therefore, we wanted to determine the phenotype of those cells by investigating the vascular and axonal infiltration in both conditions. Very little vascular infiltration into the stroke/hydrogel region was found in all three conditions. Further analysis showed a significantly higher percentage of vessels in the pen-infarct area of the MAP gel (22%) compared to both nano-porous gel and No gel conditions (6%) (FIG. 14). These results highlight the fact that different tissues have substantially different post-implantation reactions to the same material. Brain tissue remodels slower than skin tissue and will likely require other bioactive signals beyond the scaffold for revascularization such as growth factors. The increase of vessels in the peri-infarct area for MAP over nano-porous is interesting because the material has no contact with this area. This implies that the inflammatory reaction in the MAP-treated animals lead to a pro-angiogenic pen-infarct environment. To assess axonal infiltration, tissue was stained for the axonal marker NF200, which stains for the neurofilament cytoskeleton of axons and quantified the positive signal within the infarct area. No differences in axonal processes into the stroke site were observed (FIG. 15).

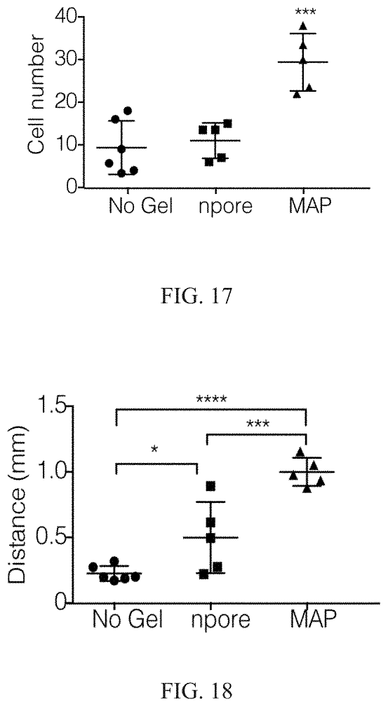

Progenitor cell migration towards the damaged tissue is a post-stroke spontaneous endogenous response to promote tissue repair. However, due to inhibitory environmental cues at the injury site these progenitor cells do not always reach the diseased tissue nor lead to tissue repair. In the brain, neural progenitor cells (NPCs) reside in the subventricular zone (SVZ) and the dentate gyrus (DG) and are activated after injury to proliferate, migrate and differentiate toward the injured tissue. NPC activation was investigated including whether the MAP hydrogel material increased their proliferation and migration from the SVZ. To identify the NPCs tissue was stained for doublecortin (DCX) and for proliferating cells using Ki67. Cells that are double positive for DCX and Ki67 are considered proliferating NPCs. Three separate analyses were performed to characterize NPC activation: the cell number along the ventricle wall (FIG. 16), the migrating cell number (FIG. 17), and the migration distance from the SVZ (FIG. 18). Upon injury, the NPC population begins to divide to self-renew. For animals injected with MAP hydrogels it was found that there was an average of 34±6 NPCs per section along the ventricle wall (FIG. 16), while the nano-porous treated animals had a significantly lower average of 18±3 NPCs, a number similar to the No Gel condition. As expected, NPCs were observed migrating along the corpus callosum towards the infarct. Almost triple the amount of proliferating NPCs were counted migrating towards the damaged tissue in the MAP gel versus the nano-porous gel and No gel condition (FIG. 17). The analysis of the migration distance from the tip of the ventricle toward the leading edge of the migrating cells revealed that the NPCs in the MAP condition migrated an average of 1 mm compared to less than 0.5 mm in the nano-porous condition (FIG. 18). No differences were observed in both the NPC cell number along the ventricle wall and the migrating cell number between the nano-porous and No Gel condition, however, in the No Gel condition, the NPCs migrated less than 0.23 mm, less than half of the nano-porous condition (FIG. 18).

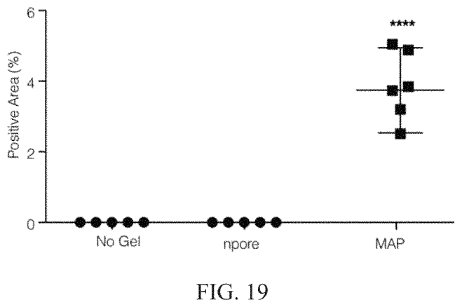

The pen-infarct and infarct areas were next examined to determine if NPCs were able to reach the infarct site at 10-days post implantation. To our surprise we observed NPCs not only in the pen-infarct area but also in the infarct area of the MAP hydrogel condition only. Indeed, no NPC migration into the stroke area was observed in both the nano-porous and the No gel conditions. This is an interesting observation as migrating NPCs into a damaged post-stroke site has never been observed before. The NPCs migrated as far as 300 μm into the MAP scaffold and occupied 3.75%±1.2 of the stroke surface as seen in FIG. 19. Interestingly, the migration pattern and distance appeared to be similar to that observed for astrocytes. Co-staining for NPC and astrocytes showed that the NPCs co-localized with the infiltrating astrocytes, suggesting that astrocyte penetration is paving a path for NPC infiltration.

The experiments establish that injectable particle-based hydrogels, namely, HA-Ac based MAP hydrogels accelerate brain repair processes by altering post-stroke astroglyosis and inflammation, changes that lead to enhanced vascularization at the pen-infarct cavity and neural progenitor cell migration within the damaged site. The present material contains hyaluronic acid, MMP, K, Q and RGD peptides as bioactive signals. Although it is likely that during the FXIIIa enzyme mediated annealing process, endogenous proteins present in the stroke cavity are incorporated into the material via the same chemistry, this is not believed to be the reason for the observed differences in inflammatory response upon material injection because FXIIIa enzyme was also added to the HA non porous condition. Rather, it is believed that the porosity of the scaffold allows for a cell infiltration into the MAP hydrogel independently of scaffold degradation. The nanoporous hydrogel contains the same bioactive components and it did not result in reduced inflammatory reaction or NPC infiltration.

While embodiments of the present invention have been shown and described, various modifications may be made without departing from the scope of the present invention. The invention, therefore, should not be limited, except to the following claims, and their equivalents.

1. A hydrogel material for the treatment of stroke or other brain injury comprising:
a collection of hyaluronic acid-based microgel particles of non-uniform size comprising one or more crosslinker components for linking different microgel particles, wherein the hyaluronic acid-based microgel particles, when exposed to an endogenous or exogenous annealing agent, links the hyaluronic acid-based microgel particles together in situ to form a covalently-stabilized scaffold of microgel particles having interstitial spaces therein.

2. The hydrogel material of claim 1, wherein the endogenous annealing agent comprises Factor XIIIa.

3. The hydrogel material of claim 1, wherein the exogenous annealing agent comprises Factor III and thrombin.

4. The hydrogel material of claim 1, wherein the collection of hyaluronic acid-based microgel particles have a diameter within the range from about 20 μm to about 120 μm.

5. The hydrogel material of claim 1, wherein the covalently-stabilized scaffold of microgel particles has a void fraction of around 10%.

6. The hydrogel material of claim 1, wherein the covalently-stabilized scaffold of microgel particles has a stiffness substantially similar to native cortex tissue of the brain.

7. The hydrogel material of claim 1, wherein the hydrogel material comprises acrylate functionalized hyaluronic acid.

8. The hydrogel material of claim 1, further comprising an adhesion peptide.

9. The hydrogel material of claim 1, further comprising at least one of a trophic factor and a growth factor.

10. A method of using the hydrogel material of claim 1 comprising injecting the collection of hyaluronic acid-based microgel particles into a stroke cavity.

11. A method of using the hydrogel material of claim 1 comprising applying the collection of hyaluronic acid-based microgel particles into or onto brain tissue.

12. A method of treating stroke in a subject comprising:
injecting a collection of hyaluronic acid-based microgel particles of non-uniform size comprising one or more crosslinker components for linking different microgel particles into a stroke cavity, wherein the hyaluronic acid-based microgel particles, when exposed to an endogenous or exogenous annealing agent, links the hyaluronic acid-based microgel particles together in situ to form a scaffold of microgel particles having interstitial spaces therein.

13. The method of claim 12, wherein the scaffold is degradable.

14. The method of claim 12, wherein the collection of hyaluronic acid-based microgel particles are injected with one or more exogenous annealing agents.

15. The method of claim 14, wherein the exogenous annealing agents comprise Factor XIII and thrombin.

16. The method of claim 12, wherein the stroke cavity is substantially filed with the hyaluronic acid-based microgel particles.

17. The method of claim 12, wherein the collection of hyaluronic acid-based microgel particles have a diameter within the range from about 20 μm to about 120 μm.

18. The method of claim 12, wherein the scaffold of microgel particles has a void fraction of around 10%.

19. The method of claim 12, wherein the exogenous annealing agent comprises a free transfer agent and a photoinitiator and wherein the hyaluronic acid-based microgel particles are exposed to light from a light source.

20. The method of claim 12, wherein the hyaluronic acid-based microgel particles is a non-binary collection of different sizes.


SEQUENCE LISTING (4 sequences)

SEQ ID NO: 1 - protein, 10 aa, Artificial Sequence
  Cell adhesive peptide
RGDSPGERCG

SEQ ID NO: 2 - protein, 8 aa, Artificial Sequence
  Linker molecule
FKGGERCG

SEQ ID NO: 3 - protein, 14 aa, Artificial Sequence
  Linker molecule
NQEQVSPLGGERCG

SEQ ID NO: 4 - protein, 16 aa, Artificial Sequence
  matrix metalloprotease degradable crosslinker
GCRDGPQGIWGQDRCG